(19) United States
(12) Patent Application Publication
(10) Pub. No.: US 2024/0415590 A1
(43) Pub. Date: Dec. 19, 2024

(54) SURGEON CONTROL OF ROBOT MOBILE CART AND SETUP ARM

(71) Applicant: Covidlen LP, Mansfield, MA (US)

(72) Inventors: Colin H. Murphy, CAmbridge, MA (US); William J. Peine, Ashland, MA (US); Kevin R. Fogarty, New York, NY (US)

(21) Appl. No.: 18/700,794

(22) PCT Filed: Nov. 17, 2022
(86) PCT No.: PCT/IB2022/061097

§ 371 Date: Apr. 12, 2024

Related U.S. Application Data
(60) Provisional application No. 63/281,182, filed on Nov. 19, 2021.

(51) Int. Cl. A61B 34/37; A61B 34/00; A61B 34/20; A61B 50/13; G16H 40/67

(52) U.S. Cl. CPC A61B 34/37; A61B 34/20; A61B 34/25; A61B 34/74; A61B 50/13; G16H 40/67

(57) ABSTRACT

A method for a surgeon to control a mobile cart, setup arm, or robotic arm of a surgical robotic system, which enables the surgeon to make adjustments without scrubbing into the bedside (a sterile environment) from the surgeon console (a non-sterile environment). The method also includes using analytics and intra-operative guidance to inform the surgeon how to adjust the components of the surgical robotic system to improve efficiency in preparing the system for optimal performance.

### BACKGROUND

Surgical robotic systems may include a surgeon console controlling one or more surgical robotic arms, each having a surgical instrument having an end effector (e.g., forceps or grasping instrument). In operation, the robotic arm is moved to a position over a patient and the surgical instrument is guided into a small incision via a surgical access port or a natural orifice of a patient to position the end effector at a work site within the patient's body.

### SUMMARY

This disclosure describes a robotic surgical system including features that allow the surgeon to control a mobile robotic cart having a setup arm and a robotic arm holding an instrument. In particular, the surgeon may use a graphical user interface or other controllers to remotely control the mobile cart, the setup arm, and/or the robotic arm. This may be done at any time, such as when the instrument is removed from the patient and undocked from an access port. To facilitate this feature, some, all or none of the setup arm joints, robotic arm joints, cart height joint (i.e., lift), or cart base wheels may be motorized.

According to one embodiment of the disclosure, a surgical robotic system is disclosed. The surgical robotic system includes a mobile cart, a setup arm coupled to the mobile cart, and a robotic arm coupled to the setup arm. The system also includes a surgeon console having a handle controller and a display configured to output a graphical user interface (GUI) having a graphical representation of the robotic arm. The system further includes a controller configured to move at least one of the mobile cart, the setup arm, or the robotic arm based on a user input entered through at least one of the GUI or the handle controller.

Implementations of the above embodiment may include one or more of the following features. According to one aspect of the above embodiment, the controller may be further configured to receive procedure data including location of an access port couplable to the robotic arm. The controller may be further configured to calculate the position at least one of the mobile cart, the setup arm, or the robotic arm based on the procedure data. The robotic arm may include at least one joint. The display may be a touchscreen and the graphical representation may include the at least one joint. The user input may include moving the at least one joint on the graphical representation. The surgical robotic system may also include at least one proximity sensor and at least one camera disposed on at least one of the mobile cart, the setup arm, or the robotic arm. The GUI may be configured to display at least one of a proximity alarm or a video during movement of at least one of the mobile cart, the setup arm, or the robotic arm.

According to another embodiment of the disclosure, a surgical robotic system is disclosed and includes a robotic arm, a display configured to output a graphical user interface (GUI) having a graphical representation of the robotic arm, and a controller configured to move the robotic arm based on user input through the GUI.

Implementations of the above embodiment may include one or more of the following features. According to one aspect of the above embodiment, the controller may be further configured to receive procedure data including location of an access port couplable to the robotic arm. The controller may be further configured to calculate the position of the robotic arm based on the procedure data. The robotic arm may include at least one joint. The display may be a touchscreen and the graphical representation may include the at least one joint. The user input may include moving the at least one joint on the graphical representation. The surgical robotic system may include at least one proximity sensor and at least one camera disposed on the robotic arm. The GUI may be configured to display at least one of a proximity alarm or a video during movement the robotic arm.

According to a further embodiment of the present disclosure, a method for controlling a surgical robotic system is disclosed. The method includes displaying graphical user interface (GUI) having a graphical representation of at least one of a mobile cart, a setup arm, or a robotic arm on a display; receiving a user input adjusting the robotic arm, the user input entered through at least one of the GUI or a handle controller of a surgeon console; and moving at least one of the mobile cart, the setup arm, or the robotic arm based on the user input.

Implementations of the above embodiment may include one or more of the following features. According to one aspect of the above embodiment, the method may include receiving procedure data including location of an access port couplable to the robotic arm. The method may also include calculating position at least one of the mobile cart, the setup arm, or the robotic arm based on the procedure data. The method may further include detecting a physical obstacle using a proximity sensor disposed on at least one of the mobile cart, the setup arm, or the robotic arm during movement of at least one of the mobile cart, the setup arm, or the robotic arm. The method may additionally include capturing a video using a camera disposed on at least one of the mobile cart, the setup arm, or the robotic arm during movement of at least one of the mobile cart, the setup arm, or the robotic arm. The method may further include displaying at least one of a proximity alarm or a video during movement of at least one of the mobile cart, the setup arm, or the robotic arm.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of mobile carts of FIG. 1 positioned about a surgical table according to an embodiment of the present disclosure;

FIG. 6 is a schematic view of a graphical user interface for controlling a mobile cart and a surgical robotic arm of FIG. 1 according to an embodiment of the present disclosure; and

FIG. 7 is a flow chart of a method according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

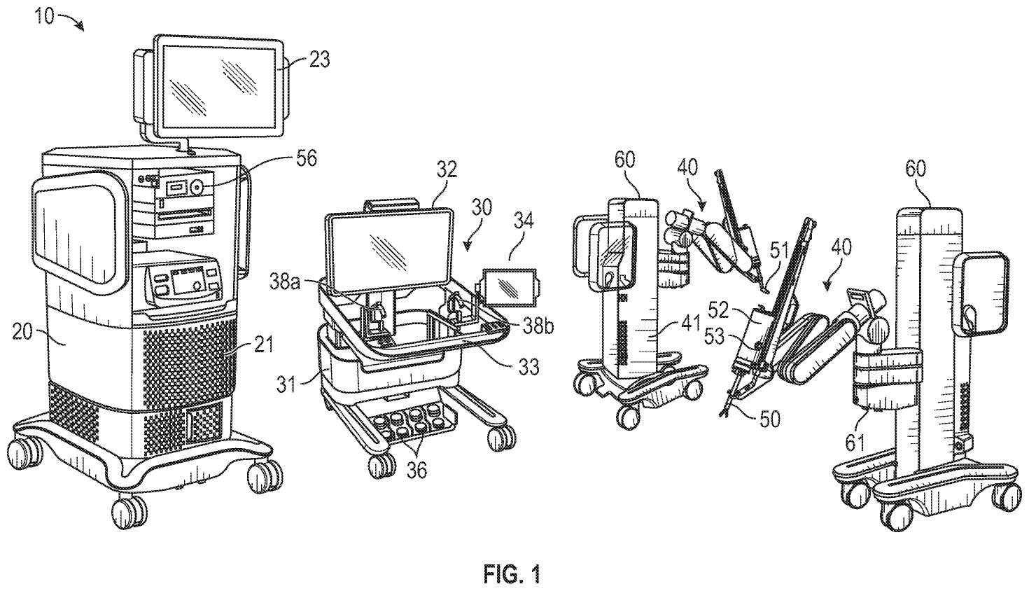

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arm 40 is also coupled to the movable cart 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgeon console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support a user's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and/or the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

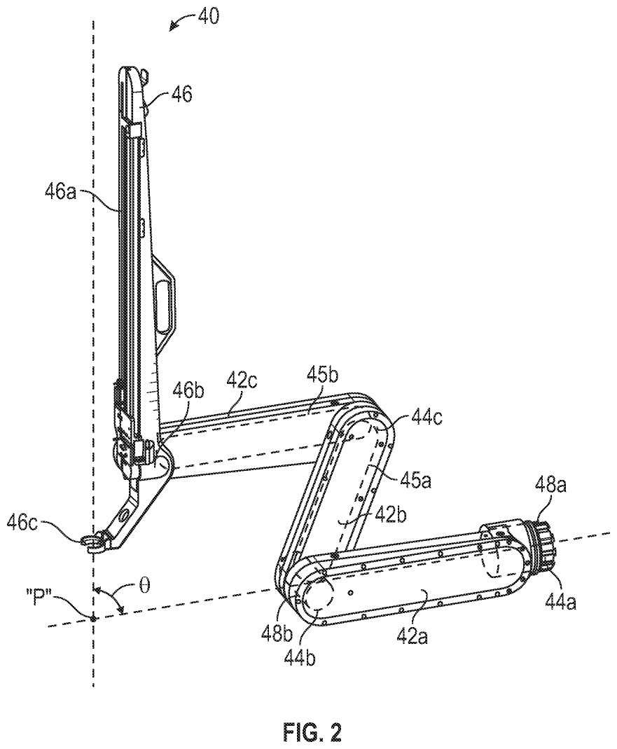

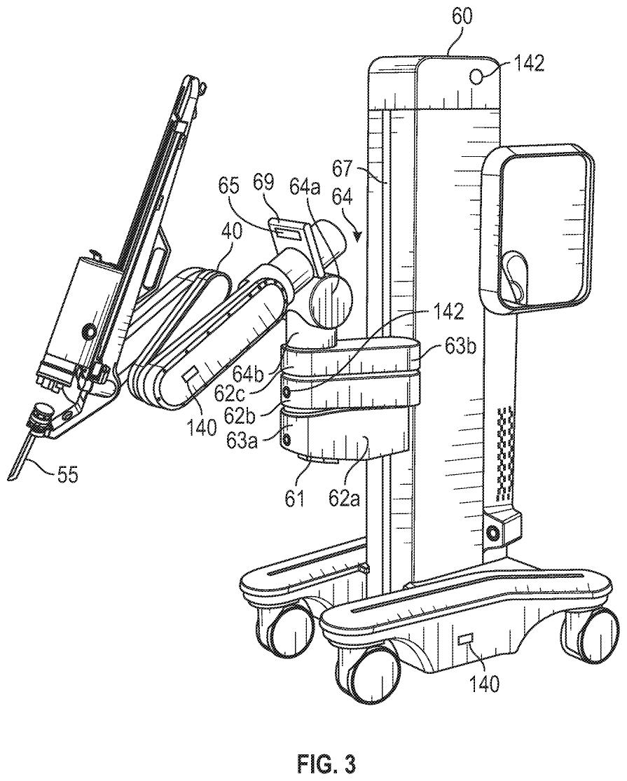

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. Joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46. The holder 46 also includes a port latch 46c for securing the port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

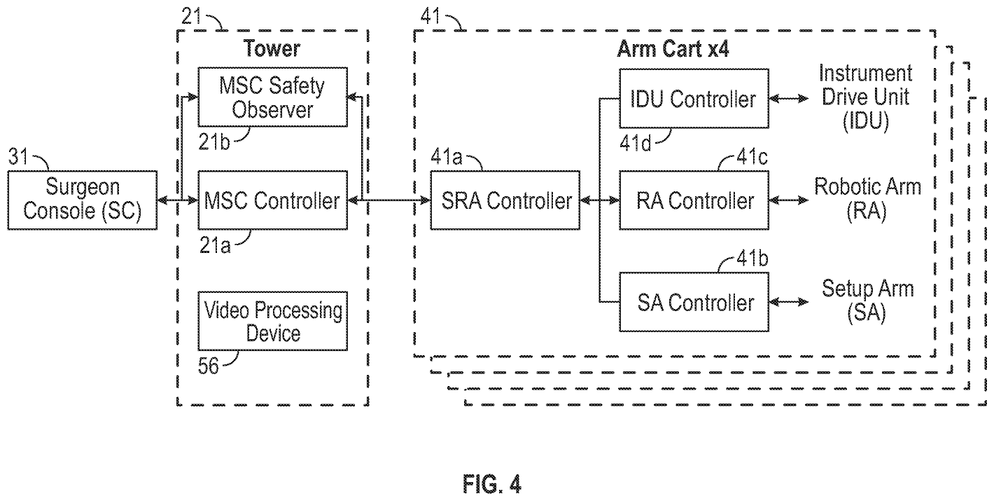

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of handle controller 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

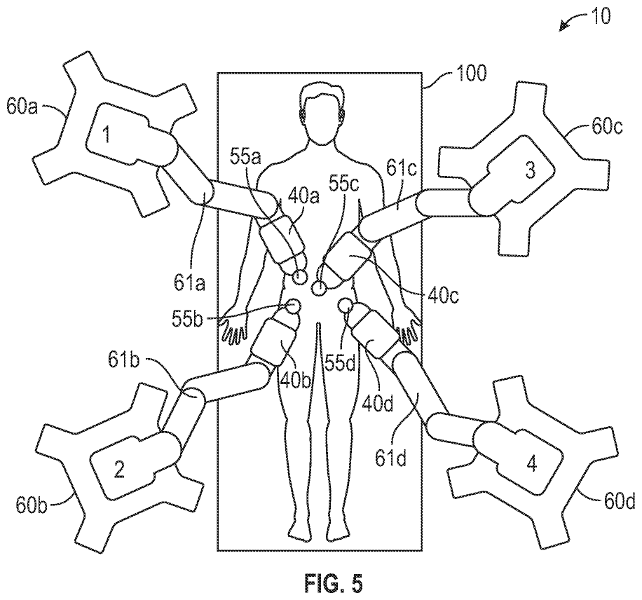

With reference to FIG. 5, the surgical robotic system 10 is setup around the surgical table 100. The system 10 includes mobile carts 60a-d, which may be numbered “1” through “4.” The mobile carts 60a-d may be positioned relative to the surgical table 100 and each other using any suitable registration system or method. During setup, each of the mobile carts 60a-d is positioned around the surgical table 100. Position and orientation of the mobile carts 60a-d depends on a plurality of factors, such as placement of a plurality of ports 55a-d, which in turn, depends on the procedure being performed. Once the port placement is determined, the ports 55a-d are inserted into the patient, and carts 60a-d are positioned and aligned relative to the surgical table 100. The setup arms 61a-d and robotic arms 40a-d of each of the mobile carts 60a-d are attached to the corresponding ports 55a-d and the instruments 50 as well as the endoscopic camera 51 are inserted into corresponding ports 55a-d.

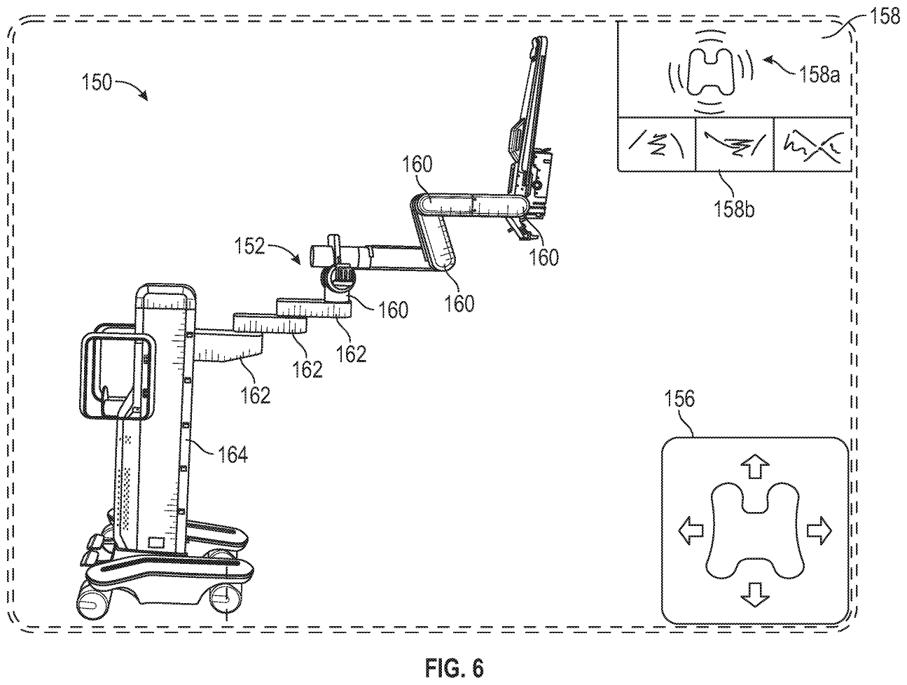

FIG. 6 shows a graphical user interface (GUI) 150 for controlling any of the mobile carts 60a-d, the setup arms 61a-d, or the robotic arms 40a-d. The mobile carts 60a-d, the setup arms 61a-d, and the robotic arms 40a-d may be moved during setup of the system 10 to minimize and/or avoid manually moving the mobile carts 60a-d relative to the surgical table 100. The GUI 150 may be displayed on any of the displays 23, 32, and 34, which are touchscreens. In addition to the GUI 150, other input devices may be used to enter movement commands such as handle controllers 38a and 38b, pedals 36, voice commands, or any other suitable controls, e.g., joystick, D-pad, etc. Furthermore, a virtual reality or augmented reality headset may be used to project the virtual mobile cart 60, setup arm 61, and robotic arm 40 onto the physical space. The virtual or augment reality projections of the robotic arm 40 and other components may be manipulated by users hands or other controllers that are registrable by cameras and/or IR projectors.

The GUI 150 displays a graphical representation 152 of the mobile cart 60, the setup arm 61, and the robotic arm 40 (FIG. 2) and includes one or more of robotic arm joints 160, one or more of setup arm joints 162, and/or a lift joint 164. The GUI 150 may display unique indicators, such as colors, numbers, etc. identifying the actual mobile cart 60, setup arm 61, and/or robotic arm 40 being controlled on the GUI 150.

The graphical representation 152 may show a 2D or 3D view of the mobile cart 60, the setup arm 61, and the robotic arm 40 and may allow for shifting of user's viewpoint, e.g., pan, rotate, zoom, etc. Each of the joints 160, 162, 164 may be controlled individually or in groups. The user may select one or more of the joints 160, 162, 164 and then issue a movement command. The GUI 150 is configured to receive input from the handle controllers 38a and 38b and/or foot pedals 36 to cycle through which joints 160, 162, 164 or groups of joints to control. In embodiments, the GUI 150 may provide selection of other control modes, such as, mobile cart 60 driving, arm approach angle movement, remote center of motion (RCM) translation, etc.

Movement commands may be entered on the GUI 150 inputting, e.g., pointing or clicking, on a desired end point, dragging a joint to a desired end point, entering coordinates, etc. Movement commands may also be entered as coordinate positions and roll-pitch-yaw (RPY) orientation relative to a coordinate reference frame (e.g., in a cartesian space of the room or in joint space). In embodiments, the RCM may be adjusted using the GUI 150 with the joints 160, 162, 164 taking the positions to achieve the commanded RCM. The RCM may be controlled via the handle controllers 38a and 38b. The graphical representation 152 may also include controls 156, e.g., arrows, for moving the mobile cart 60 relative to the surgical table 100, by activating and steering the wheels 72.

The GUI 150 is configured to display graphical representation 152 of the mobile cart 60, the setup arm 61, and/or the robotic arm 40 without moving the actual mobile cart 60, the setup arm 61, and/or the robotic arm 40 at the bedside until commanded, in order to enable the user to virtually test various configurations. Thus, the user may virtually move the mobile cart 60, the setup arm 61, and/or the robotic arm 40 and then confirm the configuration to enable movement.

The GUI 150 may output various visual indicators (e.g., color codes, alphanumeric indicators, etc.) to show the position of the joints 160, 162, 164. In embodiments, the graphical representation 152 may be updated to display the new configuration of the mobile cart 60, the setup arm 61 and/or the robotic arm 40.

Sensors and cameras may be used to aid in remote movement and adjustment of the mobile cart 60, the setup arm 61, and the robotic arm 40. With reference to FIG. 3, one or more proximity sensors 140 may be disposed on the mobile cart 60, the setup arm 61, and/or the robotic arm 40. The proximity sensors 140 may be any sensor that emits electromagnetic signal (e.g., infrared light) and measures changes in a reflected signal. The proximity sensors 140 may be used to provide feedback if the mobile cart 60, the setup arm 61, and/or the robotic arm 40 is getting close to another object.

In addition, one or more cameras 142 may be disposed on any portion of the mobile cart 60, the setup arm 61, and/or the robotic arm 40, e.g., cart base, cart column, setup arm, IDU 52 or other parts of the robotic arm 40. The cameras 142 may provide a wide-angle view of their surroundings, which when combined with the feedback from the proximity sensors 140 aids in movement of the mobile cart 60, the setup arm 61, and/or the robotic arm 40. The GUI 150 may include a region 158 displaying proximity warnings 158a along with camera views 158b, which may also be merged to provide a software-generated overhead view of the mobile cart 60, the setup arm 61, and/or the robotic arm 40.

In embodiments, the controller 21a may automate some or all movement commands of the mobile cart 60, the setup arm 61, and/or the robotic arm 40. Automatic movement may be used to move the mobile cart 60, the setup arm 61, and/or the robotic arm 40 to a desired location and/or configuration. In particular, the controller 21a may limit and/or override certain manual movement commands entered through the GUI 150 if the movement command would result in collision and/or approaching boundaries of objects, e.g., other mobile carts 60a-d. The level of automation may be adjustable by the user, and automatic control may be combined with localization of the robotic arms 40a-d relative to each other and the surgical table 100. In this case the surgeon is still controlling movement with simpler motions or commands, while the controller 21a makes more precise movement adjustments.

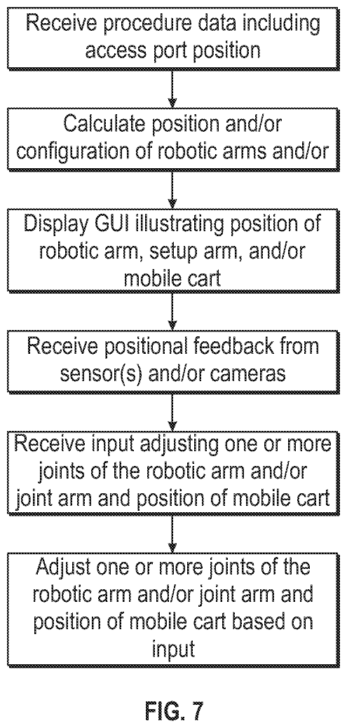

With reference to FIG. 7 a flow chart of a method for controlling movement of the mobile cart 60, the setup arm 61, and/or the robotic arm 40 includes using analytics to inform the users when an adjustment of the of the mobile cart 60, the setup arm 61, and/or the robotic arm 40 may be needed. The method may be embodied as an algorithm, which may be formulated as software instructions executed by one or more of the controllers of the system 10, e.g., the controller 21a.

Analytics may be used to aid in avoiding collisions between robotic arm 40a-d and increase dexterity of the instruments 50 by increasing the range of motion. Analytics may be based on various data, such as procedure data and internal workspace, which in turn, determines placement of access ports 55a-d. Thus, at step 200, procedure data is received by the controller 21a, which may include the position of the access ports 55a-d.

Analytics may be used to generate a desired position of the mobile cart 60, the setup arm 61, and/or the robotic arm 40. The GUI 150 may provide guidance to the surgeon by displaying a transparent view of a desired configuration and/or position which the user may use as a guide to move the mobile cart 60, the setup arm 61, and/or the robotic arm 40 to achieve the desired position. The guidance may include providing guiding lines that show an expected future position to which the mobile cart 60, the setup arm 61, and/or the robotic arm 40 is moving, and indicating if such expected movement is toward any potential collisions. The surgeon console 30 may also output auditory alarms to alert the user when using this feature to avoid collisions.

At step 202, the controller 21a calculates a position and/or location of the mobile carts 60 (see e.g., FIG. 5), including position and/or angles of each of the joints the setup arm 61, and/or the robotic arm 40. The algorithm uses localization information about where the arms are relative to each other and the patient. Joint position may be embodied as a range of motion calculation, which may be used to limit manual movement commands from the user, i.e., through the GUI 150. This feature may be used to determine when collisions may occur and at which specific joints.

The controller 21a may be connected to a cloud (i.e., one or more remote data servers), which may be used to perform more complex position and/or location calculations for the mobile carts 60a-d using a larger data set based on information collected from a plurality of surgeries previously performed by the system 10.

At step 204, as described above with respect to FIG. 6, the calculated position and/or location of the mobile carts 60a-d can be displayed to the surgeon using the GUI 150 that show the current and desired configuration mobile cart 60, the setup arm 61, and/or the robotic arm 40.

At step 206, positional feedback information from one or more of the sensors 140 and/or one or more cameras 142 is provided to the controller 21a and is displayed on the GUI 150. The feedback may be used by the user controlling the system 10 and the system 10 could also adjust the setup arm 61 and/or mobile cart 60 position automatically based on this information.

The algorithm may also enable all joints 160, 162, 164 to position themselves, automatically or with user guidance, towards a central position. The position may be user-selected or centered on a predetermined point, e.g., the camera 51. This centering facilitates improved robotic arm configuration for instrument insertion. The algorithm may also enable automated, or with user guidance, repositioning of the robotic arm 40 such that the instrument 50 will be on the display 32 after insertion. This centering also enables intra-operative adjustment of the robotic arm 40.

At step 208, the user inputs commands for moving the mobile cart 60, the setup arm 61, and/or the robotic arm 40. In embodiments, the controller 21a may act in a supervisory capacity to move the mobile cart 60, the setup arm 61, and/or the robotic arm 40 and/or adjust movement commands manually input by the user. At step 210, the manual and/or automated movement commands are provided to the mobile cart 60, the setup arm 61, and/or the robotic arm 40 to achieve the desired, i.e., commanded, configuration.

Using sensor and/or camera data about the relative positions of the mobile cart 60, the setup arm 61, and/or the robotic arm 40 and the intended procedure or internal workspace, the system 10 enables automated or surgeon-assisted exploration of the workspace of the robotic arms 40a-d. This may be done after docking the robotic arms 40a-d to the access ports 50a-d, but before inserting instruments. Moving the robotic arms 40a-d through their intended ranges of motion enables the surgeon to ensure that the risk of external arm-to-arm collisions has been minimized and/or eliminated. The surgeon may use their control of the mobile cart 60, the setup arm 61, and/or the robotic arm 40 to improve collision avoidance, or otherwise optimize internal workspace without leaving the non-sterile surgeon console.

Steps 204-210 may be repeated to adjust the robotic arms 40a-d during the procedure. Thus, if any workspace issues arise intraoperatively, (e.g., potential arm-to-arm collisions, joint range of motion, etc.), the surgeon may use the movement control to resolve such issues without the need to leave the console or for other staff to enter the sterile field. In these scenarios, the system 10 could provide feedback to the user, for instance torque and force loads on the joints, in order to help optimize location of the access ports 50a-d and port site stress.

In particular, where a collision between the robotic arms 40a-d has occurred, the surgeon can ask the bedside staff to remove the instrument 50 and undock the robotic arm 40 from the access port 50. The surgeon could then remotely control the mobile cart 60, the setup arm 61, and/or the robotic arm 40 from the surgeon console 30. This lets the surgeon move a non-sterile component while remaining outside the sterile field. Furthermore, the disclosed movement control feature may be used to drive the mobile cart 50 to reposition it in the sterile field.

The adjustment process may occur prior to teleoperation of the system 10, e.g., using instruments 50 during surgery, and may occur during setup and configuration of the system 10 or during instrument exchange. In embodiments, the GUI 150 and other control methodologies may be locked out during teleoperation and may be used only before or after teleoperation is completed.

In further embodiments, additional input controllers may be used to facilitate movement of the robotic arm 40 outside the sterile field. A miniature scale model of the robotic arm 40 may be disposed outside the sterile field that allows for manipulation of the robotic arm 40 such that movement of the miniature links, moves the links of the robotic arm 40 in a similar, albeit scaled manner. The scaled model arm includes a plurality of sensors and a plurality of movable links similar to the robotic arm 40. The sensors are configured to measure position of each of the links and provide the measurements as movement inputs to the robotic arm 40, which is then moved in the manner described above. Furthermore, the GUI 150 may be replaced by a virtual or augmented reality

It will be understood that various modifications may be made to the embodiments disclosed herein. In embodiments, the sensors may be disposed on any suitable portion of the robotic arm. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a mobile cart;
a setup arm coupled to the mobile cart;
a robotic arm coupled to the setup arm;
a surgeon console including:
a handle controller; and
a display configured to output a graphical user interface (GUI) having a graphical representation of the robotic arm; and
a controller configured to move the mobile cart and the setup arm based on a user input manually entered through the GUI or the handle controller.

2. The surgical robotic system according to claim 1, wherein the controller is further configured to receive procedure data including location of an access port couplable to the robotic arm and to calculate a position of the robotic arm based on the procedure data.

3. The surgical robotic system according to claim 2, wherein the controller is configured to couple to a remote server to receive the procedure data therefrom.

4. The surgical robotic system according to claim 1, wherein the controller is further configured to move the robotic arm based on the user input manually entered through the GUI or the handle controller and the robotic arm includes at least one joint and the graphical representation includes the at least one joint.

5. The surgical robotic system according to claim 4, the display is a touchscreen, and the user input includes moving the at least one joint on the graphical representation.

6. The surgical robotic system according to claim 1, further comprising at least one proximity sensor and at least one camera disposed on at least one of the mobile cart, the setup arm, or the robotic arm, wherein the GUI is configured to display at least one of a proximity alarm or a video during movement of the robotic arm.

7. The surgical robotic system according to claim 6, wherein the controller is configured to prevent the mobile cart and the setup arm being moved based on the user input manually entered through the GUI or the handle controller during operation of an instrument coupled to the robotic arm.

8. A surgical robotic system comprising:
a robotic arm;
a display configured to output a graphical user interface (GUI) having a graphical representation of the robotic arm; and
a controller configured to move the robotic arm based on a user input through the GUI.

9. The surgical robotic system according to claim 8, wherein the controller is further configured to receive procedure data including location of an access port couplable to the robotic arm.

10. The surgical robotic system according to claim 9, wherein the controller is further configured to calculate position the robotic arm based on the procedure data.

11. The surgical robotic system according to claim 8, wherein the robotic arm includes at least one joint.

12. The surgical robotic system according to claim 11, wherein the display is a touchscreen and the graphical representation includes the at least one joint and the user input includes moving the at least one joint on the graphical representation.

13. The surgical robotic system according to claim 8, further comprising at least one proximity sensor and at least one camera disposed on the robotic arm.

14. The surgical robotic system according to claim 13, wherein the GUI is configured to display at least one of a proximity alarm or a video during movement the robotic arm.

15. A method for controlling a surgical robotic system, the method comprising:
displaying graphical user interface (GUI) on a display of a surgeon console, the GUI being a touchscreen and having a graphical representation of at least one of a mobile cart, a setup arm, or a robotic arm of the surgical robotic system;
receiving a user input adjusting a position of the mobile cart, the setup arm, or the robotic arm, the user input entered through the GUI; and
moving at least one of the mobile cart, the setup arm, or the robotic arm based on the user input.

16. The method according to claim 15, further comprising:
receiving procedure data including location of an access port couplable to the robotic arm.

17. The method according to claim 16, further comprising:
calculating a position at least one of the mobile cart, the setup arm, or the robotic arm based on the procedure data.

18. The method according to claim 15, further comprising:
detecting a physical obstacle using a proximity sensor disposed on at least one of the mobile cart, the setup arm, or the robotic arm during movement of at least one of the mobile cart, the setup arm, or the robotic arm.

19. The method according to claim 18, further comprising:
capturing a video using a camera disposed on at least one of the mobile cart, the setup arm, or the robotic arm during movement of at least one of the mobile cart, the setup arm, or the robotic arm.

20. The method according to claim 19, further comprising:
displaying at least one of a proximity alarm or a video during movement of at least one of the mobile cart, the setup arm, or the robotic arm.
